(19) United States
(12) Patent Application Publication
(10) Pub. No.: US 2024/0379242 A1
(43) Pub. Date: Nov. 14, 2024

(54) METHOD AND SYSTEM FOR PROGNOSTIC SURVIVAL STAGE PREDICTION BASED ON MACHINE LEARNING

(71) Applicants: PEKING UNIVERSITY SCHOOL OF STOMATOLOGY, Beijing (CN); BEIHANG UNIVERSITY, Beijing (CN)

(72) Inventors: Xin PENG, Beijing (CN); Haihui WANG, Beijing (CN); Mengqi JIA, Beijing (CN); Xuechao WANG, Beijing (CN); Min GAO, Beijing (CN); Guangyan YU, Beijing (CN); Wenbo ZHANG, Beijing (CN); Wen DU, Beijing (CN); Yao YU, Beijing (CN); Peng YE, Beijing (CN)

(21) Appl. No.: 18/784,092

(22) Filed: Jul. 25, 2024

Related U.S. Application Data
(63) Continuation of application No. PCT/CN2023/072544, filed on Jan. 17, 2023.

(30) Foreign Application Priority Data
Jan. 28, 2022 (CN) 202210109421.2

(51) Int. Cl. G16H 50/30

(52) U.S. Cl. CPC G16H 50/30

(57) ABSTRACT

A method for prognostic survival stage prediction based on machine learning includes acquiring patients' original information data within a previous preset time period and integrating the patients' original information data so as to obtain a first data set without recurrence time and a second data set with recurrence time; performing analysis based on preoperative information, postoperative information and survival status of each corresponding patient, so as to obtain a correlation degree among the preoperative information, the postoperative information and the survival status; and training in the first data set, so as to obtain a postoperative survival probability prediction model. The method includes training in the second data set, so as to obtain a survival time period prediction model if judging that a survival probability of a target patient is less than or equal to a preset value according to the postoperative survival probability prediction model.

### CROSS REFERENCE

This application claims the priority of the Chinese patent application No. 202210109421.2, filed with the Chinese Patent Office on Jan. 28, 2022 and entitled “METHOD AND SYSTEM FOR PROGNOSTIC SURVIVAL STAGE PREDICTION BASED ON MACHINE LEARNING”, which is incorporated herein by reference in its entirety.

### TECHNICAL FIELD

The present application relates to the technical field of data statistics, and particularly relates to a method and a system for prognostic survival stage prediction based on machine learning.

### BACKGROUND ART

At present, surgery, radiotherapy, chemotherapy and biological therapy are four major means for cancer treatment. Taking the treatment of salivary gland cancer as an example, at present, comprehensive sequential therapy is advocated for the treatment of salivary gland cancer, i.e., according to the specific situations of patients, a variety of planned and stepwise treatment means are adopted, so as to achieve the best treatment effect. However, before the implementation of medical means, it is impossible to combine big data to give basic prognostic survival situation judgment, and it is impossible to provide more accurate prognosis result prediction for doctors and patients. Moreover, in the prior art, the preservation of patients' condition and prognosis information cannot be standardized, therefore the accumulation of historical patient data cannot be formed.

### SUMMARY OF THE INVENTION

An object of the present application is to provide a method and a system for prognostic survival stage prediction based on machine learning, so as to at least partially solve the technical problem in the prior art that the prognostic survival situation judgment cannot be made based on big data. This object is achieved through the following technical scheme.

The present application provides a method for prognostic survival stage prediction based on machine learning, including:
acquiring patients' original information data within a previous preset time period, and integrating the patients' original information data so as to obtain a first data set without recurrence time and a second data set with recurrence time, wherein each data set includes preoperative information, postoperative information and survival status of a corresponding patient;
performing analysis based on the preoperative information, the postoperative information and the survival status of each corresponding patient, so as to obtain a correlation degree among the preoperative information, the postoperative information and the survival status;
training in the first data set based on the correlation degree among the preoperative information, the postoperative information and the survival status, so as to obtain a postoperative survival probability prediction model; and
training in the second data set so as to obtain a survival time period prediction model if judging that a survival probability of a target patient is less than or equal to a preset value according to the postoperative survival probability prediction model.

Further, after performing analysis to obtain the correlation degree among the preoperative information, the postoperative information and the survival status, the method also includes:
analyzing a degree of influence of a variety of the preoperative information and a variety of the postoperative information on the survival status, in order to obtain influence degree results corresponding to multiple influence factors; and
sequencing each influence factor based on the influence degree results.

Further, analyzing the degree of influence of a variety of the preoperative information and a variety of the postoperative information on the survival status specifically includes:
analyzing the degree of influence of a variety of the preoperative information and a variety of the postoperative information on the survival status by use of the chi-square test, F-test, information gain, Pearson correlation, Spearman correlation and decision tree algorithm.

Further, performing analysis to obtain the correlation degree among the preoperative information, the postoperative information and the survival status specifically includes:
performing analysis by use of the Kaplan-Meier analysis method so as to obtain the correlation degree among the preoperative information, the postoperative information and the survival status.

Further, integrating the patients' original information data specifically includes:
dividing the preoperative information, the postoperative information and the survival status into multiple necessary features;
traversing the patients' original information data and deleting data which does not contain all the necessary features; and
preprocessing remaining data after the deleting and dividing the preprocessed data into a training set and a validation set.

Further, preprocessing the remaining data after the deleting specifically includes:
performing one-hot encoding and normalization processing on the remaining data by use of staging features and distant metastasis features, so as to obtain the training set and the validation set.

Further, a data ratio of the training set to the validation set is 9:1.

The present application also provides a system for prognostic survival stage prediction based on machine learning, wherein the system is used for implementing the above method and includes:
a data processing unit configured for acquiring the patients' original information data within the previous preset time period and integrating the patients' original information data so as to obtain the first data set without recurrence time and the second data set with recurrence time, wherein each data set includes the preoperative information, the postoperative information and the survival status of the corresponding patient;
a correlation degree analysis unit configured for performing analysis based on the preoperative information, the postoperative information and the survival status of each corresponding patient, so as to obtain the correlation degree among the preoperative information, the postoperative information and the survival status;
a first prediction model generation unit configured for training in the first data set based on the correlation degree among the preoperative information, the postoperative information and the survival status, so as to obtain the postoperative survival probability prediction model; and
a second prediction model generation unit configured for training in the second data set so as to obtain the survival time period prediction model if judging that the survival probability of the target patient is less than or equal to the preset value according to the postoperative survival probability prediction model.

The present application also provides an intelligent terminal which includes a data collector, a processor and a memory, wherein
the data collector is configured for collecting data; the memory is configured for storing one or more program instructions; and the processor is configured for executing the one or more program instructions so as to execute the above method.

The present application also provides a computer readable storage medium which includes one or more program instructions, and the one or more program instructions are configured for executing the above method.

According to the method for prognostic survival stage prediction based on machine learning provided by the present application, a prognostic survival model, which is constructed by using original data as a basis and combining with an artificial intelligence machine learning algorithm, is capable of assisting doctors to predict the prognosis of patients. Furthermore, based on statistical analysis, the factors which have an important impact on prognostic survival are obtained, and the influence degrees thereof are sequenced to ensure that the prediction accuracy of the prognosis model is higher. The technical problem in the prior art that prognostic survival situation judgment cannot be made based on big data is solved.

### BRIEF DESCRIPTION OF THE DRAWINGS

The various other advantages and beneficial effects of the present application will become apparent to those skilled in the art by reading the following detailed description of preferred embodiments. The drawings are merely for the purpose of showing the preferred embodiments, but shall not be considered as limitation to the present application. In the whole drawings, the same components are represented by the same reference numerals. In the drawings:

FIG. 1 is a flow chart of a specific implementation of the method for prognostic survival stage prediction based on machine learning provided by the present application;

FIG. 2 is a feature importance sequencing chart; and

FIG. 3 is a structural block diagram of a specific implementation of the method for prognostic survival stage prediction based on machine learning provided by the present application.

### DETAILED DESCRIPTION OF THE INVENTION

Hereafter, exemplary implementations of the present disclosure are described in more detail with reference to the drawings. Although the drawings show the exemplary implementations of the present disclosure, it is to be understood that, the present disclosure can be realized in various forms, and should not be limited to the implementations set forth herein. On the contrary, these implementations are provided to enable a more thorough understanding of the present disclosure and to fully convey the scope of the present disclosure to those skilled in the art.

The present application provides a method for prognostic survival stage prediction based on machine learning, wherein the method is capable of giving the sequence of factors affecting patients' condition more accurately, and predicting the postoperative survival of patients by stages accordingly, so as to normatively store detailed data of the patients.

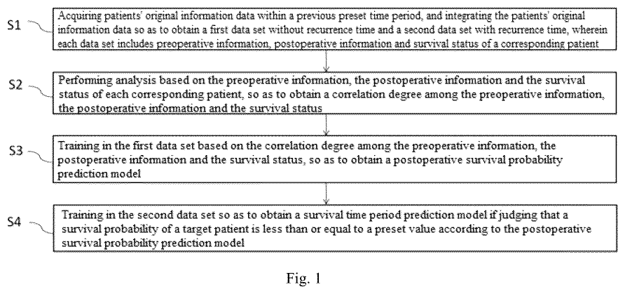

In a specific implementation, as shown in FIG. 1, the method for prognostic survival stage prediction based on machine learning provided by the present application includes the following steps.
S1: acquiring patients' original information data within a previous preset time period, and integrating the patients' original information data so as to obtain a first data set without recurrence time and a second data set with recurrence time, wherein each data set includes preoperative information, postoperative information and survival status of a corresponding patient.

The step of integrating the patients' original information data includes the following sub-steps.
S101: dividing the preoperative information, the postoperative information and the survival status into multiple necessary features which can include a variety of feature information characterizing disease types, disease pathology types and disease stages;
S102: traversing the patients' original information data and deleting data which does not contain all the necessary features; and
S103: preprocessing remaining data after the deleting and dividing the preprocessed data into a training set and a validation set. This step specifically includes: performing one-hot encoding and normalization processing on the remaining data by use of staging features and distant metastasis features, so as to obtain the training set and the validation set, wherein a data ratio of the training set to the validation set is 9:1.
S2: performing analysis based on the preoperative information, the postoperative information and the survival status of each corresponding patient, so as to obtain a correlation degree among the preoperative information, the postoperative information and the survival status. This step specifically includes: performing analysis by use of the Kaplan-Meier analysis method so as to obtain the correlation degree among the preoperative information, the postoperative information and the survival status.

Further, after performing analysis to obtain the correlation degree among the preoperative information, the postoperative information and the survival status, the method also includes:
analyzing a degree of influence of a variety of the preoperative information and a variety of the postoperative information on the survival status, in order to obtain influence degree results corresponding to multiple influence factors. This step specifically includes: analyzing the degree of influence of a variety of the preoperative information and a variety of the postoperative information on the survival status by use of the chi-square test, F-test, information gain, Pearson correlation, Spearman correlation and decision tree algorithm.

In this embodiment, the chi-square test and Pearson correlation algorithm are taken as examples for description, and other algorithms are similar thereto and will not be described repeatedly.

Specifically, chi-square test belongs to the category of non-parametric test, which is mainly used to compare two or more sample rates (constituent ratio) and perform the correlation analysis of two categorical variables. Its basic idea is to compare the degree of coincidence or goodness of fit between theoretical frequency and actual frequency. The larger the chi-square value is, the greater the deviation between the actual observed value and the expected value is, thus indicating that the mutual independence of the two events is weaker. The specific calculation formula is as follows:

$${χ}^{2} = {\sum }_{} \frac{{( A - E )}^{2}}{E} = {\sum }_{i = 1}^{k} \frac{{( {A}_{i} - {E}_{i} )}^{2}}{{E}_{i}} = {\sum }_{i = \{ 1}^{k} \frac{{( {A}_{i} - {np}_{i} )}^{2}}{{np}_{i}}$$

Pearson correlation is also a method for measuring the similarity of variables. It is utilized to measure the correlation degree between continuous variables, and its output ranges from −1 to +1, wherein 0 means no correlation, a negative value means negative correlation, and a positive value means positive correlation. Variables which are more similar to the target variables are considered to be more important. The specific calculation formula is as follows:

$$ρ ( X , Y ) = \frac{E [ ( X - {μ}_{X} ) ( Y - {μ}_{Y} ) ]}{{σ}_{X} {σ}_{Y}} = \frac{E [ ( X - {μ}_{X} ) ( Y - {μ}_{Y} ) ]}{\sqrt{{\sum }_{i = 1}^{n} {( {X}_{i} - {μ}_{X} )}^{2}} \sqrt{{\sum }_{i = 1}^{n} {( {Y}_{i} - {μ}_{Y} )}^{2}}}$$

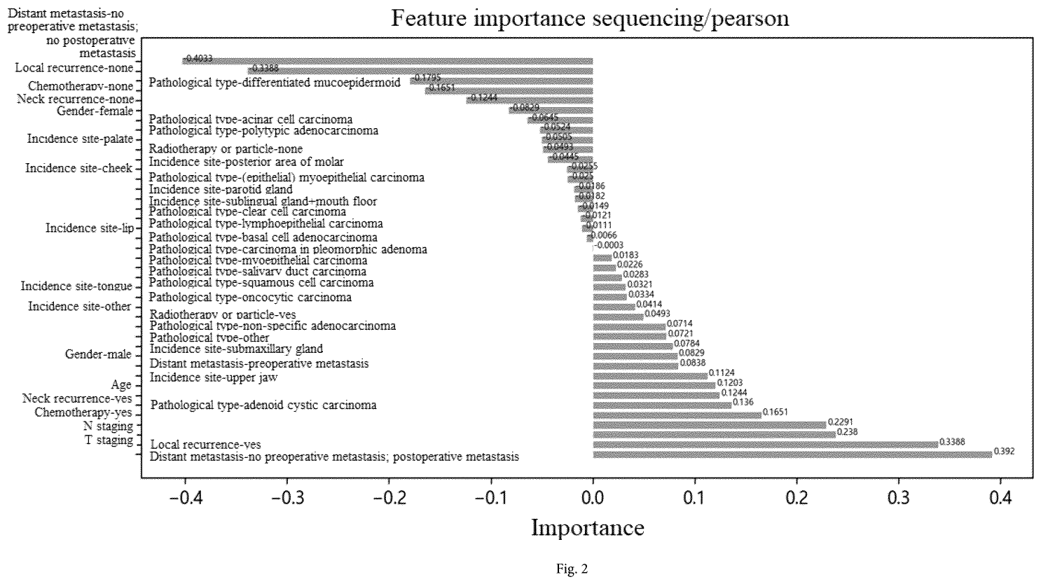

In this embodiment, an influence factor analysis is performed on the target variable “patient survival status”. An influence factor having a strong correlation with this target variable is of high importance, whereas an influence factor having a low correlation with this target variable is of low importance. A sequencing operation on influence factors based on influence degree results is specifically shown in FIG. 2:
S3: training in the first data set based on the correlation degree among the preoperative information, the postoperative information and the survival status, so as to obtain a postoperative survival probability prediction model; and
S4: training in the second data set so as to obtain a survival time period prediction model if judging that a survival probability of a target patient is less than or equal to a preset value according to the postoperative survival probability prediction model.

In the above specific implementation, according to the method for prognostic survival stage prediction based on machine learning provided by the present application, a prognostic survival model, which is constructed by using original data as a basis and combining with an artificial intelligence machine learning algorithm, is capable of assisting doctors to predict the prognosis of patients. Furthermore, based on statistical analysis, the factors which have an important impact on prognostic survival are obtained, and the influence degrees thereof are sequenced to ensure that the prediction accuracy of the prognosis model is higher. The technical problem in the prior art that prognostic survival situation judgment cannot be made based on big data is solved.

Hereinafter, the establishment of a salivary gland cancer prognosis model is taken as an example to briefly describe the specific implementing process of the method for prognostic survival stage prediction provided by the present application.

Salivary gland cancer is one of the most common malignant tumors of the head and neck, and its occurrence is related to a variety of internal and external factors, including smoking, drinking, viral infection, malnutrition, dietary habits, local stimulation, and the like, wherein especially smoking and drinking are the most harmful. From a worldwide perspective, oral and pharyngeal cancers with higher incidence rate are the sixth most common malignant tumor in the body (ranking behind lung cancer, stomach cancer, breast cancer, colon and rectal cancers, and cervical cancer), with about 350,000 to 400,000 new cases each year. China has a large population, and the actual number of cases of salivary gland cancer ranked among the top in the world.

The following step is included in predicting the prognosis of salivary gland cancer patients by use of the method provided by the present application:
S100: acquiring the patients' original information data within a previous preset time period, and integrating the patients' original information data.

This step is utilized to perform enhancement processing on the original patient data.

Firstly, a global analysis is performed on the original data, wherein the original data includes two data sets which are respectively “a data set without recurrence time” and “a data set with recurrence time”. Each data set is composed of three parts, namely “preoperative information”, “postoperative information” and “survival status” of a patient.

Features of the “preoperative information” may include gender; age; incidence sites, such as parotid gland, submaxillary gland, sublingual gland, palate, posterior area of molar, check, tongue, lip, upper jaw and other parts; pathological types, such as well differentiated mucoepidermoid carcinoma, moderately differentiated mucoepidermoid carcinoma, poorly differentiated mucoepidermoid carcinoma, adenoid cystic carcinoma, carcinoma in pleomorphic adenoma, non-specific adenocarcinoma, acinar cell carcinoma, myoepithelial carcinoma, polytypic adenocarcinoma, basal cell adenocarcinoma, salivary duct carcinoma, squamous cell carcinoma, lymphoepithelial carcinoma, epithelial-myoepithelial carcinoma, oncocytic carcinoma, clear cell carcinoma and other types; T staging which is divided into stages 1, 2, 3, and 4 according to the size and spread range of the primary tumor; N staging which is divided into grades 0, 1, 2 and 3 according to the size, texture and adhesion situation of lymph nodes; and M staging, such as determining whether preoperative distant metastasis occurs according to various clinical examination results.

Features of the “postoperative information” may include follow-up time, such as the interval between the last follow-up time and the date of surgery on a monthly basis; local recurrence, such as whether postoperative recurrence occurs in the primary site; neck recurrence, such as whether postoperative cervical metastasis occurs; distant metastasis, such as whether postoperative distant metastasis occurs, wherein if preoperative metastasis occurs, regardless of whether postoperative distant metastasis occurs, it is labeled as metastasis; radiotherapy, such as whether postoperative radiotherapy or particle radiotherapy is supplemented, including none, yes or unknown; and chemotherapy, such as whether postoperative chemotherapy is supplemented, including none, yes or unknown.

Features of the “survival status” include survival status, such as tumor-free survival; the tumor is removed completely without recurrence, and the patient is in survival status; survival with tumor; the tumor is not removed completely, and the patient is still in survival status; recurrence and death; the tumor recurrence at the primary site leads to patient death; metastatic death; the tumor metastasis to other sites, such as the lung, brain, bone, etc., leads to patient death; other causes of patient death, such as cerebral hemorrhage, car accidents, suicide, and other cancers; and all-cause death, such as the patients' survival status at the end of follow-up, including: survival, death from salivary gland malignancy, and death from other diseases.

The above features are the information features which needed to be included in the first data set. Further, the second data set with recurrence time is obtained by adding patient recurrence time to the part of postoperative information of patients on the basis of the first data set without recurrence time. The corresponding features are changed as follows: local recurrence, such as whether postoperative recurrence occurs in the primary site, wherein “\” represents no recurrence, the number represents recurrence and is the recurrence time, unit: month; neck recurrence, such as whether postoperative cervical metastasis occurs, wherein “\” represents no recurrence, the number represents recurrence and is the recurrence time, unit: month; and distant metastasis, such as whether postoperative distant metastasis occurs, wherein “\” represents no metastasis, the number represents metastasis and is the metastasis time, unit: month.

After the data sets are sorted and classified by use of the above method, the distribution and data integrity of each feature in the data sets, such as gender, age, incidence sites, pathological types, T staging, N staging, M staging, follow-up time, local recurrence, neck recurrence, distant metastasis, radiotherapy; chemotherapy, survival status and all-cause death, are analyzed. The Python programming language is adopted to draw and visually display the data. Since the data integrity reaches 97.9%, the data with incomplete feature information is directly deleted.

Then, the remaining data is preprocessed. The specific process is as follows:
Step 1: selecting information of 13 features, namely “gender”, “age”, “incidence sites”, “pathological types”, “T staging”, “N staging”, “M staging”, “local recurrence”, “neck recurrence”, “distant metastasis”, “radiotherapy”, “chemotherapy” and “all-cause death”, from the original data;
Step 2: deleting the “unknown” patient data from the “radiotherapy” or “chemotherapy” feature information;
Step 3: deleting the patient data with “other causes of death” from the “survival status” feature information;
Step 4: refining the “distant metastasis” feature information by use of the “M staging” feature information, wherein this step is specifically as follows: if the “M staging” feature information is “no preoperative metastasis” and the “distant metastasis” feature information is “no postoperative metastasis” for a salivary gland cancer patient, labelling the “distant metastasis” feature information of the patient as “distant metastasis-no preoperative metastasis; no postoperative metastasis”; if the “M staging” feature information is “no preoperative metastasis” and the “distant metastasis” feature information is “postoperative metastasis” for a salivary gland cancer patient, labelling the “distant metastasis” feature information of the patient as “distant metastasis-no preoperative metastasis; postoperative metastasis”; and if the “M staging” feature information is “preoperative metastasis” for a salivary gland cancer patient, labelling the “distant metastasis” feature information of the patient as “distant metastasis-preoperative metastasis”;
Step 5: deleting the “M staging” feature information;
Step 6: performing one-hot encoding on the “gender”, “incidence sites”, “pathological types”, “local recurrence”, “neck recurrence”, “distant metastasis”, “radiotherapy” and “chemotherapy” feature information;
Step 7: performing max-min normalization processing on the “T staging”, “N staging” and “age” feature information;
Step 8: dividing the preprocessed data set into a training set and a validation set at a ratio of 9:1;
Step 9: checking whether the distribution states of the respective feature information of the training set and a test set are roughly the same.

Sequencing of important influence factors exemplarily includes the following steps:
firstly, the Kaplan-Meier analysis method is used to analyze the correlation degree between each feature and the prognostic survival status of a patient;
secondly, the degree of influence of each feature information on the patient's prognosis is analyzed and processed by use of the chi-square test, F-test, information gain, Pearson correlation, Spearman correlation and decision tree algorithm;
and finally, analysis results of various methods are integrated by means of a “voting method”, so as to provide the comprehensive influence factor sequence.

Establishment of a patient prognosis model exemplarily includes the following steps:
training a machine learning integrated algorithm LightGBM-model by use of the feature information in the first data set according to the available actual data, thus obtaining a postoperative survival probability prediction model; and
training the machine learning integrated algorithm LightGBM-model by use of the patients' postoperative time information in the second data set, thus obtaining a survival time period prediction model.

In practical application, the postoperative survival probability prediction model is responsible for predicting in the first stage, and providing the patient's postoperative survival probability with the accuracy rate more than 91%; if the prediction result obtained by the postoperative survival probability prediction model shows that the survival probability of the target patient is less than 50%, the survival time period prediction model is responsible for predicting the second stage, and providing the probabilities of the survival time of the target patient in the three time periods of “less than 2 years”, “2 years to 5 years” and “more than 5 years”. The detailed information of patients is saved according to the existing historical data format so as to form standardized data accumulation.

It can be seen from the foregoing that, in the case of salivary gland cancer, according to the present application, a salivary gland cancer patient prognostic survival model is constructed by means of relying on oral medicine as the background and combining with the artificial intelligence machine learning algorithm. For the historical salivary gland cancer patient data, in terms of the statistics, machine learning algorithms and product limit methods aspects, the factors which have an important impact on the postoperative survival of salivary gland cancer patients are concluded and summarized by use of the voting method, and the influence degrees thereof are sequenced, thus making the prognosis prediction more targeted. Meanwhile, according to the method, various index information and detailed postoperative follow-up time information in the salivary gland cancer patient data are utilized to respectively train the postoperative survival probability prediction model and the survival time period prediction model for patients' prognosis, and the accuracy rate of overall prediction is more than 91% on the premise of ensuring the robustness of the models. In practical application, according to the method, the postoperative survival prediction is carried out on salivary gland cancer patients in stages, and meanwhile, automatic and standardized storage for patient condition information and prognosis information is carried out, thus forming historical patient data accumulation.

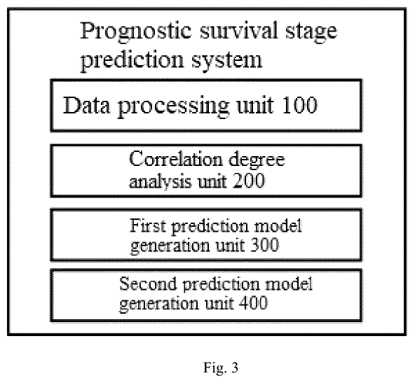

In addition to the above method, the present application also provides a system for prognostic survival stage prediction based on machine learning, wherein the system is used for implementing the above method. In a specific embodiment, as shown in FIG. 3, the system includes:
a data processing unit 100 used for acquiring the patients' original information data within a previous preset time period and integrating the patients' original information data so as to obtain a first data set without recurrence time and a second data set with recurrence time, wherein each data set includes preoperative information, postoperative information and survival status of a corresponding patient;
a correlation degree analysis unit 200 used for performing analysis based on the preoperative information, the postoperative information and the survival status of each corresponding patient, so as to obtain a correlation degree among the preoperative information, the postoperative information and the survival status;
a first prediction model generation unit 300 used for training in the first data set based on the correlation degree among the preoperative information, the postoperative information and the survival status, so as to obtain a postoperative survival probability prediction model; and a second prediction model generation unit 400 used for training in the second data set so as to obtain a survival time period prediction model if judging that a survival probability of a target patient is less than or equal to a preset value according to the postoperative survival probability prediction model.

In the above specific implementations, according to the system for prognostic survival stage prediction based on machine learning provided by the present application, a prognostic survival model, which is constructed by using original data as a basis and combining with an artificial intelligence machine learning algorithm, is capable of assisting doctors to predict the prognosis of patients. Furthermore, based on statistical analysis, the factors which have an important impact on prognostic survival are obtained, and the influence degrees thereof are sequenced to ensure that the prediction accuracy of the prognosis model is higher. The technical problem in the prior art that prognostic survival situation judgment cannot be made based on big data is solved.

The present application also provides an intelligent terminal which includes a data collector, a processor and a memory.

The data collector is used for collecting data; the memory is used for storing one or more program instructions; and the processor is used for executing the one or more program instructions so as to execute the above method.

In corresponding to the above embodiment, the present application also provides a computer storage medium, and the computer storage medium includes one or more program instructions, wherein the one or more program instructions are used for executing the method by a depth calibration system for binocular cameras.

It is to be understood that, the terms used herein are merely for the purpose of describing specific exemplary implementations, but are not intended to limit the present application. Unless otherwise clearly specified in the context, the singular forms “a”, “an”, and “the” used herein may also include the plural forms. The terms “comprise”, “include”, “contain”, and “have” are inclusive and thus indicate the existence of the stated features, steps, operations, elements and/or components, but do not exclude the existence or addition of one or more other features, steps, operations, elements, components and/or combinations thereof. The method steps, procedures and operations described herein are not interpreted as requiring that they must be performed in a specific sequence as described or illustrated, unless the sequence of execution is explicitly specified. It should also be understood that additional or alternative steps can be used.

Although the terms such as first, second and third can be used herein to describe multiple elements, components, regions, layers and/or segments, these elements, components, regions, layers and/or segments should not be limited by these terms. These terms can be merely used for distinguishing one element, component, region, layer or segment from another region, layer or segment. Unless otherwise clearly specified in the context, the terms such as “first” and “second” and other numerical terms used herein do not imply the sequence or order. Therefore, the first element, component, region, layer or segment discussed hereafter can be referred to as the second element, component, region, layer or segment without departing from the teaching of the exemplary implementations.

In the embodiment of the present application, the processor can be an integrated circuit chip with signal processing capability. The processor can be a general-purpose processor, a Digital Signal Processor (DSP), an Application Specific Integrated Circuit (ASIC), a Field Programmable Gate Array (FPGA) or other programmable logic devices, discrete gate or transistor logic devices, discrete hardware components.

The methods, steps and logical block diagrams disclosed in the embodiments of the present application can be realized or executed. The general-purpose processor can be a microprocessor or the processor can be any conventional processor, etc. The steps of the methods disclosed in combination with the embodiments of the present application may be directly embodied as being completely executed by a hardware decoding processor or by the combination of hardware and software modules in a decoding processor. Software modules can be located in a mature storage medium such as a random memory, a flash memory; a read-only memory, a programmable read-only memory or an electrically erasable programmable memory, a register and the like in the field. The processor reads the information in the storage medium, and completes the steps of the above method in combination with hardware thereof.

The storage medium can be a memory, such as a volatile memory or a non-volatile memory, or can include both the volatile memory and the non-volatile memory.

The non-volatile memory can be a Read-Only Memory (ROM), a Programmable ROM (PROM), an Erasable PROM (EPROM), an Electrically EPROM (EEPROM), or a flash memory.

The volatile memory can be a Random Access Memory (RAM), which is used as an external cache. By way of exemplary description instead of restrictive description, many forms of RAMs are available, such as a Static RAM (SRAM), a Dynamic RAM (DRAM), a Synchronous DRAM (SDRAM), a Double Data Rate SDRAM (DDRSDRAM), an Enhanced SDRAM (ESDRAM), a Synchlink DRAM (SLDRAM) and a DirectRambus RAM (DRRAM).

The storage medium described in the embodiments of the present application is intended to include but not be limited to these and any other suitable type of memories.

Those skilled in the art should be aware of that, in one or more of the foregoing examples, the functions described in the present application can be realized by use of a combination of hardware and software. When software is applied, corresponding functions can be stored in a computer-readable medium or transmitted as one or more instructions or codes on the computer-readable medium. The computer-readable medium includes a computer storage medium and a communication medium, wherein the communication medium includes any medium which facilitates transmission of a computer program from one place to another place. The storage medium can be any available medium which can be accessed by a general-purpose or special-purpose computer.

The foregoing specific implementations are provided for further detailed description on the purpose, technical scheme and beneficial effects of the present application. It should be understood that, the foregoing is merely specific implementations of the present application and is not intended to limit the protection scope of the present application. Any modification, equivalent replacement, improvement, etc. made on the basis of the technical scheme of the present application shall be included within the protection scope of the present application.

### INDUSTRIAL APPLICABILITY

The present application provides a method and a system for prognostic survival stage prediction based on machine learning. According to the method and the system, a prognostic survival model, which is constructed by using original data as a basis and combining with an artificial intelligence machine learning algorithm, is capable of assisting doctors to predict the prognosis of patients. Furthermore, based on statistical analysis, the factors which have an important impact on prognostic survival are obtained, and the influence degrees thereof are sequenced to ensure that the prediction accuracy of the prognosis model is higher. The technical problem in the prior art that prognostic survival situation judgment cannot be made based on big data is solved.

1. A method for prognostic survival stage prediction based on machine learning, comprising:
acquiring patients' original information data within a previous preset time period and integrating the patients' original information data so as to obtain a first data set without recurrence time and a second data set with recurrence time, wherein each data set comprises preoperative information, postoperative information and survival status of a corresponding patient;
performing analysis based on the preoperative information, the postoperative information and the survival status of each corresponding patient, so as to obtain a correlation degree among the preoperative information, the postoperative information and the survival status;
training in the first data set based on the correlation degree among the preoperative information, the postoperative information and the survival status, so as to obtain a postoperative survival probability prediction model; and
training in the second data set so as to obtain a survival time period prediction model if judging that a survival probability of a target patient is less than or equal to a preset value according to the postoperative survival probability prediction model.

2. The method for prognostic survival stage prediction according to claim 1, wherein after performing analysis to obtain the correlation degree among the preoperative information, the postoperative information and the survival status, the method further comprises:
analyzing a degree of influence of a variety of the preoperative information and a variety of the postoperative information on the survival status, in order to obtain influence degree results corresponding to multiple influence factors; and
sequencing the influence factors based on the influence degree results.

3. The method for prognostic survival stage prediction according to claim 2, wherein analyzing the degree of influence of a variety of the preoperative information and a variety of the postoperative information on the survival status specifically comprises:
analyzing the degree of influence of a variety of the preoperative information and a variety of the postoperative information on the survival status by use of the chi-square test, F-test, information gain, Pearson correlation, Spearman correlation and decision tree algorithm.

4. The method for prognostic survival stage prediction according to claim 2, wherein performing analysis to obtain the correlation degree among the preoperative information, the postoperative information and the survival status specifically comprises:
performing analysis by use of the Kaplan-Meier analysis method so as to obtain the correlation degree among the preoperative information, the postoperative information and the survival status.

5. The method for prognostic survival stage prediction according to claim 1, wherein integrating the patients' original information data specifically comprises:
dividing the preoperative information, the postoperative information and the survival status into multiple necessary features;
traversing the patients' original information data and deleting data which does not contain all the necessary features; and
preprocessing remaining data after the deleting and dividing the preprocessed data into a training set and a validation set.

6. The method for prognostic survival stage prediction according to claim 5, wherein preprocessing the remaining data after the deleting specifically comprises:
performing one-hot encoding and normalization processing on the remaining data by use of staging features and distant metastasis features, so as to obtain the training set and the validation set.

7. The method for prognostic survival stage prediction according to claim 6, wherein a data ratio of the training set to the validation set is 9:1.

8. A system for prognostic survival stage prediction based on machine learning, which is used for implementing the method according to claim 1 and comprises:
a data processing unit configured for acquiring the patients' original information data within the previous preset time period and integrating the patients' original information data so as to obtain the first data set without recurrence time and the second data set with recurrence time, wherein each data set comprises the preoperative information, the postoperative information and the survival status of the corresponding patient;
a correlation degree analysis unit configured for performing analysis based on the preoperative information, the postoperative information and the survival status of each corresponding patient, so as to obtain the correlation degree among the preoperative information, the postoperative information and the survival status;
a first prediction model generation unit configured for training in the first data set based on the correlation degree among the preoperative information, the postoperative information and the survival status, so as to obtain the postoperative survival probability prediction model; and
a second prediction model generation unit configured for training in the second data set so as to obtain the survival time period prediction model if judging that the survival probability of the target patient is less than or equal to the preset value according to the postoperative survival probability prediction model.

9. An intelligent terminal, comprising a data collector, a processor and a memory, wherein
the data collector is configured for collecting data; the memory is configured for storing one or more program instructions; and the processor is configured for executing the one or more program instructions so as to execute the method according to claim 1.

10. A computer readable storage medium, comprising one or more program instructions, and the one or more program instructions are configured for executing the method according to claim 1.
